(19) United States
(12) Patent Application Publication
(10) Pub. No.: US 2010/0076022 A1
(43) Pub. Date: Mar. 25, 2010

(54) NOVEL POLYMORPHS OF RIMONABANT

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Kesireddy Subash Chander Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 11/993,566

(22) PCT Filed: Sep. 1, 2006
(86) PCT No.: PCT/IN06/00331

§ 371 Date: Dec. 21, 2007

(51) Int. Cl. C07D 401/12; A61K 31/454

(52) U.S. Cl. 514/326; 546/211

(57) ABSTRACT

The present invention discloses novel and stable polymorphs of rimonabant, its hydrates and solvates, to the processes for their preparation and to pharmaceutical compositions comprising them. The present invention further discloses a novel and stable amorphous form of rimonabant, process for its preparation and a pharmaceutical composition comprising it. The present invention also provides an improved process for the preparation of rimonabant crystalline Form II. Thus, for example, rimonabant is dissolved in methylene dichloride, stirred for 10 minutes at 25-30° C. and then the solvent distilled off under vacuum at 40° C. The resulting residue is stirred with water and the separated solid is collected at 25-30° C. to give a stable crystalline rimonabant hydrate.

### FIELD OF THE INVENTION

The present invention relates to novel and stable polymorphs of rimonabant, its hydrates and solvates, to the processes for their preparation and to pharmaceutical compositions comprising them. The present invention also relates to a novel and stable amorphous form of rimonabant, process for its preparation and to a pharmaceutical composition comprising it. The present invention also provides an improved process for preparation of rimonabant crystalline Form II.

### BACKGROUND OF THE INVENTION

U.S. Pat. Nos. 5,624,941 and 5,462,960 disclosed pyrazole-3-carboxamide derivatives, processes for their preparation, pharmaceutical compositions in which they are present and uses thereof. These compounds possess a very good affinity to the cannabinoid receptor and are useful in the therapeutic areas in which cannabis is known to be involved. The therapeutic indications of cannabinoids concern a variety of areas such as the immune system, the central nervous system and the cardiovascular or endocrine system. Among them, rimonabant, chemically 5-(4-chlorophenyl)-1-(2,4-dichlorophenyl)-4-methyl-N-(piperidin-1-yl)pyrazole-3-carboxamide is a promising CB₁ receptor antagonist with potent and selective activity in binding and functional assays, and which has been shown to inhibit motivational and consummatory aspects of feeding and reduce alcohol and nicotine intake in animal models. Rimonabant is represented by the following structure:

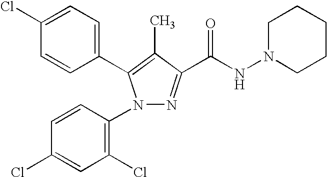

Rimonabant can exist in different crystalline forms, which differ from each other in terms of stability, physical properties, spectral data and methods of preparation.

The U.S. Pat. No. 5,624,941 makes no reference to the existence of specific polymorphic forms of rimonabant. In this patent, it is disclosed that the compound is isolated according to conventional techniques; more precisely, according to the embodiments exemplified, the product is obtained after crystallization from isopropyl ether or by cooling of a medium containing the product in methylcyclohexane. The ‘941’ patent further disclosed an ethanol solvate of rimonabant, together with the process of preparation.

U.S. Patent Appl. No. 2005/0043356 A1 described two crystalline forms of rimonabant (Form I and Form II), characterizes them by single crystal X-ray analysis, powder X-ray diffraction, infra-red spectroscopy, and differential enthalpic analysis. The U.S. Patent Appl. No. 2005/0043356 A1 further described that the synthetic procedure described and exemplified in U.S. Pat. No. 5,624,941 produces the rimonabant crystalline form designated herein as Form I (characterized by an X-ray powder diffraction pattern having peaks expressed as 2θ at about 9.1, 11.6, 12.3, 16.0, 16.4, 16.8, 18.3, 19.4, 20.7, 21.2, 22.9 and 27.2±0.1 degrees).

According to the U.S. Patent Appl. No. 2005/0043356 A1, rimonabant crystalline Form II (characterized by an X-ray powder diffraction pattern having peaks expressed as 2θ at about 5.0, 10.1, 10.7, 15.1, 19.1 and 25.4±0.1 degrees) can be prepared by dissolving rimonabant in the hot state in a solvent chosen from methylcyclohexane in the pure state or containing 1 to 10% of water by volume, acetonitrile, 4-methyl-2-pentanone, acetone or a mixture of these solvents; where appropriate, cooling the medium to a temperature of between 5° C. and 25° C.; and filtering the crystals formed at a temperature of between 5° C. and 25° C.

We have discovered a novel and highly stable crystalline hydrate form of rimonabant which differs from each of the prior art forms (Form I & Form II), in their stability, in their physical properties, in their spectral characteristics and in their method of preparation. The novel crystalline rimonabant hydrate is stable over time and has good flow properties and so, the novel crystalline hydrate is suitable for formulating rimonabant.

An amorphous form of rimonabant has not been reported in the prior art. It is well known that pharmaceutical products in amorphous form usually have better dissolution characteristics than when they are in crystalline form. So, there is a need for a stable amorphous form of rimonabant for better pharmaceutical preparations. The existence of an amorphous form of rimonabant has now been discovered. The novel amorphous rimonabant form is highly stable and found to have a better dissolution rate. So, the novel amorphous form is suitable for pharmaceutical preparations.

The present invention further discloses two stable solvated forms of rimonabant, i.e., rimonabant n-propanol solvate and rimonabant n-butanol solvate.

The n-propanol and n-butanol solvates are non-hygroscopic, obtainable in pure form and can be converted to rimonabant and its salts.

The novel solvates are useful as intermediates for preparing pure rimonabant or pharmaceutically acceptable salts of rimonabant.

One object of the present invention is to provide a stable and novel crystalline hydrate of rimonabant, process for preparing it and a pharmaceutical composition comprising it.

Another object of the present invention is to provide a stable and novel amorphous form of rimonabant, process for preparing it and a pharmaceutical composition comprising it.

Another object of the present invention is to provide rimonabant n-pronol solvate and rimonabant n-butanol solvate, and processes for preparing the solvates.

Another object of the present invention is to provide an improved process for the preparation of rimonabant crystalline Form II.

### DETAILED DESCRIPTION OF THE INVENTION

According to one aspect of the present invention, there is provided a crystalline hydrate form of rimonabant having a water content in the range of about 3-15% by weight, characterized by peaks in the powder X-ray diffraction pattern having 2θ angle positions at about 9.3, 10.5, 13.5, 14.5, 15.3, 16.1, 17.1, 17.8, 20.8, 21.1, 22.4, 22.9, 23.6 and 27.3±0.1 degrees. The typical X-ray powder diffraction pattern is shown in FIG. 1.

According to another aspect of the present invention, a process is provided for preparation of a crystalline hydrate form of rimonabant having a water content in the range of about 3-15% by weight, which comprises:
a) distilling off the solvent from a solution of rimonabant in methylene dichloride at least until precipitation of rimonabant occurs;
b) separating the solid rimonabant, if necessary;
c) slurrying the solid rimonabant in water; and
d) collecting the crystalline rimonabant hydrate having a water content in the range of about 3-15% by weight from the contents.

The solution of rimonabant used in step (a) may be obtained by dissolving rimonabant in the solvent at an ambient temperature. The rimonabant used may be in the form of rimonabant in a non-solvated form or solvated form. The solution of rimonabant obtained as part of the synthesis of rimonabant may also be used in step (a).

The distillation of the solvent may be carried out at atmospheric pressure or at reduced pressure. The distillation of the solvent may be carried out just until precipitation of rimonabant starts forming or the distillation may be carried out until substantial precipitation occurs. The distillation may also preferably be carried out until the solvent is almost completely distilled off.

The separation of the precipitated solid rimonabant in step (b) may be carried by the methods known in the art such as filtration or centrifugation.

The solid collected is slurried in water. The temperature at which slurrying is done is not critical and the slurrying may conveniently be carried out at about 20° C. to 80° C.

The crystalline rimonabant hydrate is collected from the slurry by conventional methods such as filtration or centrifugation.

The water content of crystalline rimonabant hydrate obtained by the process as described above is preferably between 3% and 12% by weight, more preferably between 3% and 6% by weight and still more preferably between 3.5% and 5.5% by weight.

The crystalline rimonabant hydrate obtained by the process as described above has a water content in the range of about 3-15% by weight, and crystalline rimonabant hydrate shows the same characteristic powder X-ray diffraction pattern throughout this water content range.

According to another aspect of the present invention, a process is provided for crystalline rimonabant hydrate having a water content in the range of about 3-15% by weight, which comprises:
a) dissolving rimonabant in methanol or acetone;
b) adding water to the solution obtained in step (a); and
c) isolating the crystalline rimonabant hydrate having a water content in the range of about 3-15% by weight from the contents.

The rimonabant may be dissolved, if necessary, at an elevated temperature. The isolation may be initiated by any conventional method usually known in the art such as cooling, seeding, partial removal of the solvent from the solution, by adding an anti-solvent to the solution or a combination thereof.

The crystalline rimonabant hydrate obtained in step (c) is collected by filtration or centrifugation.

The water content of crystalline rimonabant hydrate obtained by the process as described above is preferably between 3% and 12% by weight, more preferably between 3% and 6% by weight and still more preferably between 3.5% and 5.5% by weight.

According to another aspect of the present invention, a process is provided for crystalline rimonabant hydrate having a water content in the range of about 3-15% by weight, which comprises:
a) suspending rimonabant hydrochloride in water;
b) adjusting the pH of the above suspension to above 8.0 with a base; and
c) isolating the crystalline rimonabant hydrate having a water content in the range of about 3-15% by weight from the contents.

Preferably the pH of the suspension in the step (b) is adjusted to 8-11 and more preferably to 9.5-10.5.

A preferable base used in step (b) is an inorganic base such as liquor ammonia, sodium hydroxide and sodium bicarbonate, and a more preferable inorganic base is liquor ammonia.

The isolation may be initiated by any conventional method usually known in the art such as cooling, seeding, partial removal of the solvent from the solution, by adding an anti-solvent to the solution or a combination thereof.

The crystalline rimonabant hydrate obtained in step (c) is collected by filtration or centrifugation.

The water content of crystalline rimonabant hydrate obtained by the process as described above is preferably between 3% and 12% by weight, more preferably between 3% and 6% by weight and still more preferably between 3.5% and 5.5% by weight.

According to another aspect of the present invention, there is provided a novel amorphous form of rimonabant. The amorphous rimonabant is characterized by having a broad X-ray diffraction spectrum as in FIG. 2.

According to another aspect of the present invention, a process is provided for the preparation of amorphous rimonabant. Amorphous rimonabant is prepared by dissolving rimonabant in a solvent selected from the group consisting of an alcoholic solvent, a ketonic solvent, an ester solvent, an ether solvent, a chlorinated hydrocarbon solvent and an hydrocarbon solvent, and then removing the solvent from the solution by vacuum drying, spray drying or freeze drying.

A preferable alcoholic solvent is selected from methanol, ethanol, isopropyl alcohol, tert-butyl alcohol and n-butyl alcohol, and a more preferable alcoholic solvent is methanol or ethanol. A preferable ketonic solvent is selected from acetone, methyl isobutyl ketone and methyl ethyl ketone, and more a preferable ketonic solvent is acetone. A preferable ester solvent is ethyl acetate. A preferable ether solvent is diisopropyl ether. A preferable chlorinated hydrocarbon solvent is methylene dichloride. A preferable hydrocarbon solvent is toluene.

The rimonabant may be dissolved in a solvent at an elevated temperature, if necessary, at the reflux temperature of the solvent used. The rimonabant used may be in the form of rimonabant in a non-solvated form or a solvated form or a hydrated form. A most preferable solvent used in the above process is ethyl acetate. The solvent may preferably be removed from the solution by vacuum drying or spray drying.

According to another aspect of the present invention, there is provided a crystalline rimonabant n-propanol solvate, characterized by an X-ray powder diffraction pattern having peaks expressed as 2θ at about 6.7, 8.3, 11.9, 13.4, 14.3, 15.9, 16.5, 17.9, 18.1, 19.2, 19.8, 20.5, 20.8, 21.4, 21.8, 22.2, 22.6, 24.1, 27.0 and 28.2±0.1 degrees. FIG. 3 shows a typical X-ray powder diffraction pattern of rimonabant n-propanol solvate.

According to another aspect of the present invention, a process is provided for the preparation of crystalline rimonabant n-propanol solvate, which comprises:
a) preparing a solution of rimonabant in n-propanol; and
b) isolating the rimonabant n-propanol solvate from the solution obtained in step (a).

The solution of rimonabant is usually prepared at an elevated temperature, preferably at reflux temperature.

The isolation may be initiated by any conventional method usually known in the art such as cooling, seeding, partial removal of the solvent from the solution, by adding an anti-solvent to the solution or a combination thereof.

The solution is cooled preferably to 0° C. to 30° C. The precipitated rimonabant n-propanol solvate crystals are collected by filtration or centrifugation.

The content of n-propanol in the crystalline rimonabant n-propanol solvate obtained by the process as described above is preferably between 10% and 15% by weight.

The rimonabant n-propanol solvate is obtained in pure form, is non-hygroscopic in nature and can be converted to rimonabant or pharmaceutically acceptable salts of rimonabant in pure form.

According to another aspect of the present invention, there is provided a crystalline rimonabant n-butanol solvate, characterized by an X-ray powder diffraction pattern having peaks expressed as 2θ at about 7.5, 8.0, 9.1, 10.4, 16.1, 17.3, 22.4 and 23.8±0.1 degrees. FIG. 4 shows typical X-ray powder diffraction pattern of rimonabant n-butanol solvate.

According to another aspect of the present invention, a process is provided for the preparation of crystalline rimonabant n-butanol solvate, which comprises:
a) preparing a solution of rimonabant in n-butanol; and
b) isolating the rimonabant n-butanol solvate from the solution obtained in step (a).

The solution of rimonabant is usually prepared at an elevated temperature, preferably at reflux temperature.

The isolation may be initiated by any conventional method usually known in the art such as cooling, seeding, partial removal of the solvent from the solution, by adding an anti-solvent to the solution or a combination thereof.

The solution is cooled preferably to about 0° C. to 30° C. The precipitated rimonabant n-butanol solvate crystals are collected by filtration or centrifugation.

The content of n-butanol in the crystalline rimonabant n-butanol solvate obtained by the process as described above is preferably between about 10% and 15% by weight.

The rimonabant n-butanol solvate is obtained in pure form, is non-hygroscopic in nature and can be converted to rimonabant or pharmaceutically acceptable salts of rimonabant in pure form.

According to another aspect of the present invention, a process is provided for the preparation of rimonabant crystalline Form II, which comprises:
a) preparing a solution of rimonabant in isopropyl alcohol; and
b) isolating the rimonabant crystalline Form II from the solution obtained in step (a).

The solution of rimonabant is usually prepared at an elevated temperature, preferably at reflux temperature.

The isolation may be initiated by any conventional method usually known in the art such as cooling, seeding, partial removal of the solvent from the solution, by adding an anti-solvent to the solution or a combination thereof.

The solution is cooled preferably to about 0° C. to 30° C. The precipitated rimonabant Form II crystals are collected by filtration or centrifugation.

The content of n-butanol in the crystalline rimonabant n-butanol solvate obtained by the process as described above is preferably between about 10% and 15% by weight.

The rimonabant n-butanol solvate is obtained in pure form, is non-hygroscopic in nature and can be converted to rimonabant or pharmaceutically acceptable salts of rimonabant in pure form.

According to another aspect of the present invention, a process is provided for the preparation of rimonabant crystalline Form II, which comprises:
c) preparing a solution of rimonabant in isopropyl alcohol; and
d) isolating the rimonabant crystalline Form II from the solution obtained in step (a).

The solution of rimonabant is usually prepared at an elevated temperature, preferably at reflux temperature.

The isolation may be initiated by any conventional method usually known in the art such as cooling, seeding, partial removal of the solvent from the solution, by adding an anti-solvent to the solution or a combination thereof.

The solution is cooled preferably to about 0° C. to 30° C. The precipitated rimonabant Form II crystals are collected by filtration or centrifugation.

According to another aspect of the present invention, there is provided a pharmaceutical composition comprising crystalline rimonabant hydrate and a pharmaceutically acceptable excipient.

A preferable pharmaceutical composition of crystalline rimonabant hydrate is a solid oral dosage form.

According to another aspect of the present invention, there is provided a pharmaceutical composition comprising amorphous rimonabant and a pharmaceutically acceptable excipient.

A preferable pharmaceutical composition of amorphous rimonabant is a solid oral dosage form.

### BRIEF DESCRIPTION OF THE DRAWINGS

FIG. 1 is an X-ray powder diffraction pattern of crystalline rimonabant hydrate of the invention obtained as per the procedures described in examples 1, 2, 3 and 4.

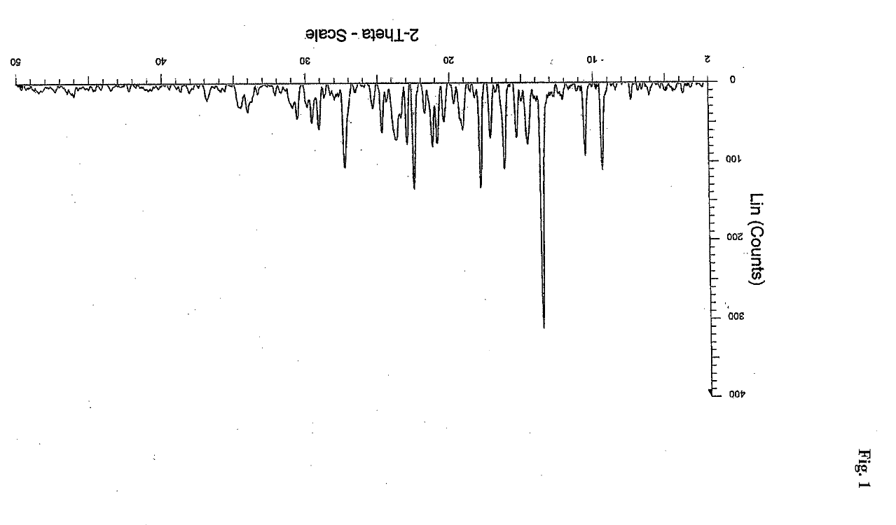

FIG. 2 is an X-ray powder diffraction pattern of amorphous rimonabant.

FIG. 3 is an X-ray powder diffraction pattern of crystalline rimonabant n-propanol solvate.

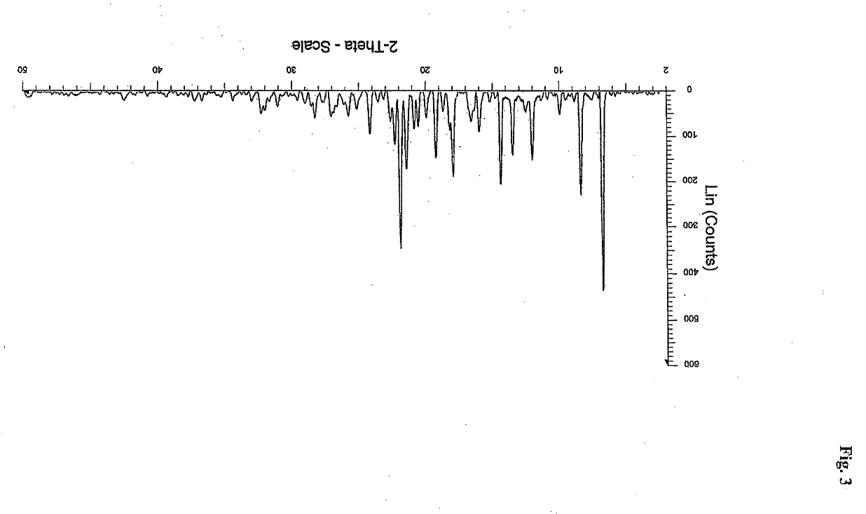

FIG. 4 is an X-ray powder diffraction pattern of crystalline rimonabant n-butanol solvate.

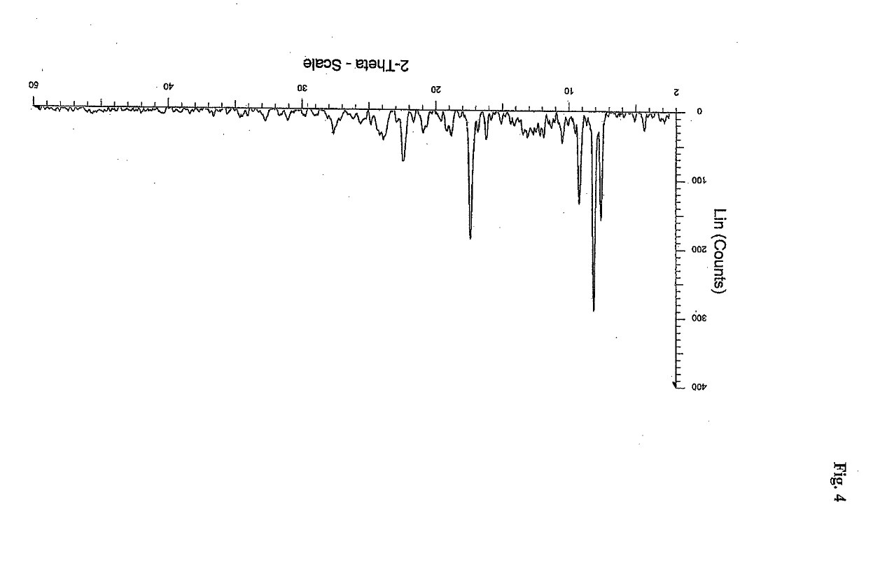

FIG. 5 is an X-ray powder diffraction pattern of crystalline rimonabant Form II obtained as per the procedure described in example 8.

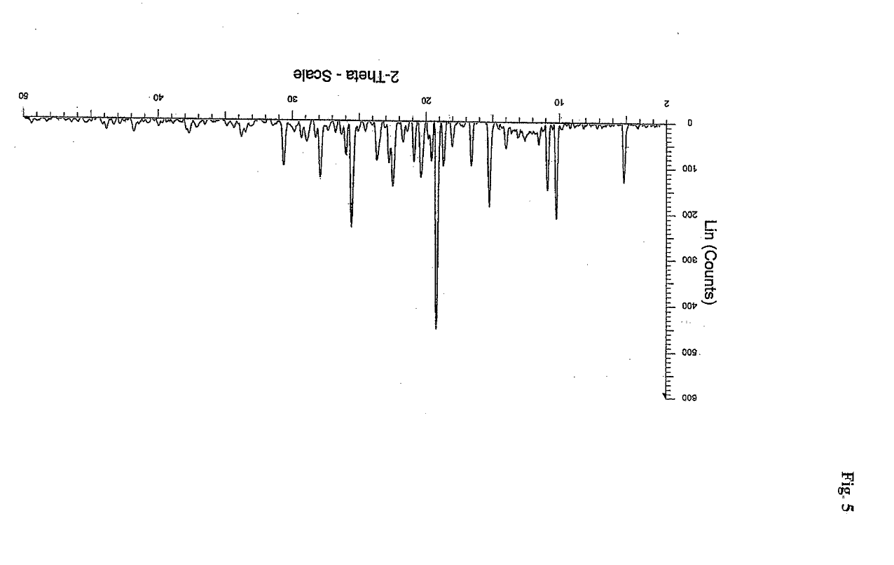

FIG. 6 shows the X-ray powder diffraction patterns of crystalline rimonabant hydrate, rimonabant crystalline Form I and Form II.

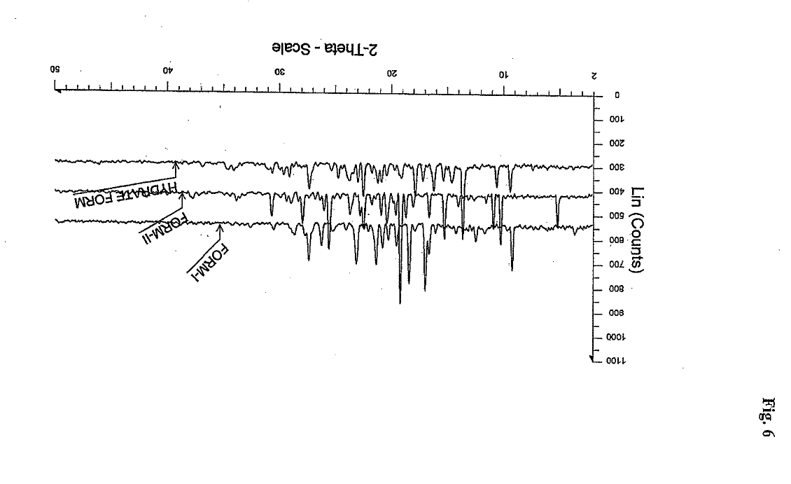

X-ray powder diffraction spectrum was measured on a bruker axs D8 advance X-ray powder diffractometer having a copper-k_{α} radiation. Approximately 1 gm of sample was gently flattened on a sample holder and scanned from 2 to 50 degrees two-theta, at 0.03 degrees two-theta per step and a step time of 0.5 seconds. The sample was simply placed on the sample holder. The sample was rotated at 30 rpm at a voltage 40 KV and 35 mA.

The following examples are given for the purpose of illustrating the present invention and should not be considered as a limitation on the scope or spirit of the invention.

### Example 1

Rimonabant (10 gm) is dissolved in methylene dichloride (25 ml) at 25-30° C., stirred for 10 minutes at 25-30° C. and then the solvent distilled off under vacuum at 40° C. To the residue is added water (20 ml) and stirred for 1 hour at 25-30° C. The solid is filtered, washed with water (5 ml) and then the material dried at 55-60° C. to give 9.5 gm of crystalline rimonabant hydrate (Moisture content: 3.8% by weight).

### Example 2

Rimonabant (10 gm) is added to acetone (60 ml) under stirring at 25-30° C., the contents are heated to 50° C. to form a clear solution and then stirred for 4 hours at 25-30° C. To the reaction mass is added water (20 ml) at 25-30° C. and stirred for 2 hours. The solid is filtered, washed with water (5 ml) and then the material dried at 50-55° C. to give 8.5 gm of crystalline rimonabant hydrate (Moisture content: 4.1% by weight).

### Example 3

Rimonabant (10 gm) is added to methanol (60 ml) under stirring at 25-30° C., the contents are heated to 55° C. to form a clear solution and then water (1 ml) is added to the solution at 55° C. The reaction mass is stirred for 2 hours at 25-30° C., the solid filtered, is washed with methanol (10 ml) and then the material dried at 50-55° C. to give 9.6 gm of crystalline rimonabant hydrate (Moisture content: 3.7% by weight).

### Example 4

Rimonabant hydrochloride (10 gm) is suspended in water (70 ml) at 25-30° C., the pH of the suspension is adjusted to 10.0 with 1.8 ml of liq. NH₃ at 25-30° C. and then stirred for 2 hours at 25-30° C. while maintaining the pH above 8.0. The solid is filtered, washed with water (10 ml) and then the material dried at 55-60° C. to give 8.9 gms of crystalline rimonabant hydrate (Moisture content: 3.9% by weight).

### Example 5

Rimonabant (10 gm) is added to ethyl acetate (60 ml) under stirring at 25-30° C., the contents are heated to 50° C. to form a clear solution and then stirred for 4 hours at 25-30° C. The reaction mass is distilled under vacuum at 45° C. and then dried at 50-55° C. to give 9.4 gm of amorphous rimonabant.

### Example 6

Rimonabant (10 gm) is added to n-propanol (60 ml) under stirring at 25-30° C., the contents are heated to 50° C. to form a clear solution and then stirred for 4 hours at 25-30° C. The reaction mass is cooled to 5° C. and stirred for 1 hour at 5-10° C. The solid is filtered, washed with n-propanol (5 ml) and then the material dried at 65-70° C. to give 9.3 gm of crystalline rimonabant n-propanol solvate (n-propanol content: 11.4% by weight).

### Example 7

Rimonabant (10 gm) is added to n-butanol (60 ml) under stirring at 25-30° C., the contents are heated to 50° C. to form a clear solution and then stirred for 4 hours at 25-30° C. The reaction mass is cooled to 5° C. and stirred for 1 hour at 5-10° C. The solid is filtered, washed with n-butanol (5 ml) and then the material dried at 65-70° C. to give 8.8 gm of crystalline rimonabant n-butanol solvate (n-butanol content: 13.7% by weight).

### Example 8

Rimonabant (10 gm) is added to isopropyl alcohol under stirring at 25-30° C., the contents are heated to 50° C. to form a clear solution and then stirred for 36 hours at 25-30° C. The solid is filtered, washed with isopropyl alcohol (10 ml) and then the material dried at 60-65° C. to give 9.3 gm of rimonabant crystalline Form II.

We claim:

1. A crystalline rimonabant hydrate having a water content in the range of about 3-15% by weight, characterized by peaks in a powder X-ray diffraction pattern having 2θ angle positions at about 9.3, 10.5, 13.5, 14.5, 15.3, 16.1, 17.1, 17.8, 20.8, 21.1, 22.4, 22.9, 23.6 and 27.3±0.1 degrees.

2. A process for the preparation of crystalline rimonabant hydrate of claim 1, which comprises:
a) distilling off solvent from a solution of rimonabant in methylene dichloride at least until precipitation of rimonabant occurs;
b) separating the solid rimonabant, if necessary;
c) slurrying the solid rimonabant in water; and
d) collecting the crystalline rimonabant hydrate having a water content in the range of about 3-15% by weight from the slurry.

3. The process as claimed in claim 2, wherein the solution of rimonabant used in step (a) is obtained by dissolving rimonabant in the solvent at an ambient temperature.

4. The process as claimed in claim 2, wherein the distillation of the solvent is carried out at atmospheric pressure or at reduced pressure.

5. The process as claimed in claim 2, wherein the distillation of the solvent is carried out just until precipitation of rimonabant starts forming or the distillation may be carried out until substantial precipitation occurs.

6. The process as claimed in claim 2, wherein the distillation of the solvent is carried out until the solvent is almost completely distilled off.

7. The process as claimed in claim 2, wherein the separation of the precipitated solid rimonabant in step (b) is carried out by filtration or centrifugation.

8. The process as claimed in claim 2, wherein the slurrying with water in step (c) is carried out at about 20° C. to 80° C.

9. The process as claimed in claim 2, wherein the crystalline rimonabant hydrate in step (d) is collected by filtration or centrifugation.

10. The process as claimed in claim 2, wherein the water content of crystalline rimonabant hydrate obtained is between 3% and 12% by weight.

11. The process as claimed in claim 10, wherein the water content of crystalline rimonabant hydrate is between 3% and 6% by weight.

12. The process as claimed in claim 11, wherein the water content of crystalline rimonabant hydrate is between 3.5% and 5.5% by weight.

13. A process for the preparation of crystalline rimonabant hydrate of claim 1, which comprises:
a) dissolving rimonabant in methanol or acetone;
b) adding water to the solution obtained in step (a); and
c) isolating the crystalline rimonabant hydrate having a water content in the range of about 3-15% by weight from the contents.

14. The process as claimed in claim 13, wherein the rimonabant is dissolved at an elevated temperature.

15. The process as claimed in claim 13, wherein the isolation is initiated by cooling, seeding, partial removal of the solvent from the solution, by adding an anti-solvent to the solution or a combination thereof.

16. The process as claimed in claim 13, wherein the crystalline rimonabant hydrate obtained in step (c) is collected by filtration or centrifugation.

17. The process as claimed in claim 13, wherein the water content of crystalline rimonabant hydrate obtained is between 3% and 12% by weight.

18. The process as claimed in claim 17, wherein the water content of crystalline rimonabant hydrate is between 3% and 6% by weight.

19. The process as claimed in claim 18, wherein the water content of crystalline rimonabant hydrate is between 3.5% and 5.5% by weight.

20. A process for the preparation of crystalline rimonabant hydrate of claim 1, which comprises:
a) suspending rimonabant hydrochloride in water;
b) adjusting the pH of the above suspension to above 8.0 with a base; and
c) isolating the crystalline rimonabant hydrate having a water content in the range of about 3-15% by weight from the contents.

21. The process as claimed in claim 20, wherein the pH of the suspension in step (b) is adjusted to 8-11.

22. The process as claimed in claim 21, wherein the pH of the suspension is adjusted to 9.5-10.5.

23. The process as claimed in claim 20, wherein the base used in step (b) is an inorganic base such as liquor ammonia, sodium hydroxide and sodium bicarbonate.

24. The process as claimed in claim 23, wherein the inorganic base is liquor ammonia.

25. The process as claimed in claim 20, wherein the isolation is initiated by cooling, seeding, partial removal of the solvent from the solution, by adding an anti-solvent to the solution or a combination thereof.

26. The process as claimed in claim 20, wherein the crystalline rimonabant hydrate obtained in step (c) is collected by filtration or centrifugation.

27. The process as claimed in claim 20, wherein the water content of crystalline rimonabant hydrate obtained is between 3% and 12% by weight.

28. The process as claimed in claim 27, wherein the water content of crystalline rimonabant hydrate is between 3% and 6% by weight.

29. The process as claimed in claim 28, wherein the water content of crystalline rimonabant hydrate is between 3.5% and 5.5% by weight.

30. Amorphous rimonabant.

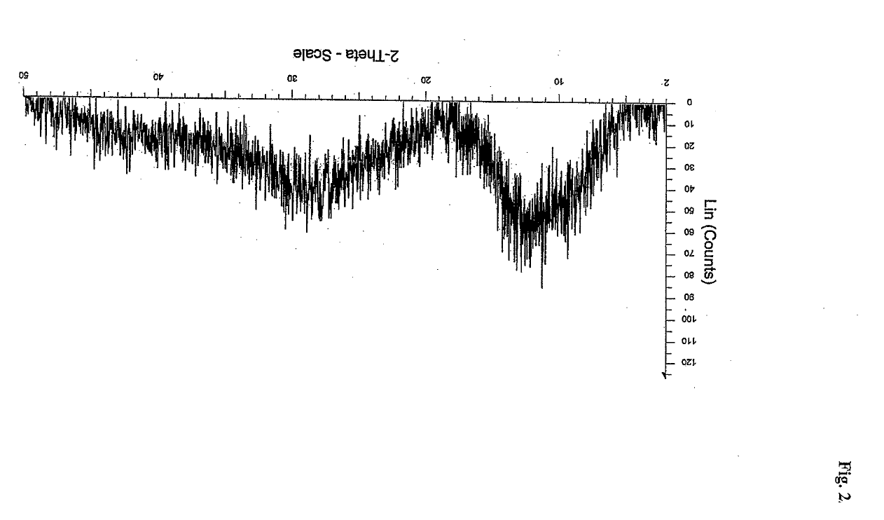

31. The compound as claimed in claim 30, wherein the amorphous rimonabant is characterized by an x-ray powder diffraction spectrum as in FIG. 2.

32. A process for the preparation of amorphous rimonabant of claim 30, which comprises dissolving rimonabant in a solvent selected from the group consisting of an alcoholic solvent, a ketonic solvent, an ester solvent, an ether solvent, a chlorinated hydrocarbon solvent and an hydrocarbon solvent, and then removing the solvent from the solution by vacuum drying, spray drying or freeze drying.

33. The process as claimed in claim 32, wherein the alcoholic solvent is selected from methanol, ethanol, isopropyl alcohol, tert-butyl alcohol and n-butyl alcohol.

34. The process as claimed in claim 33, wherein the alcoholic solvent is methanol.

35. The process as claimed in claim 32, wherein the ketonic solvent is selected from acetone, methyl isobutyl ketone and methyl ethyl ketone.

36. The process as claimed in claim 35, wherein the ketonic solvent is acetone.

37. The process as claimed in claim 32, wherein the ester solvent is ethyl acetate.

38. The process as claimed in claim 32, wherein the ether solvent is diisopropyl ether.

39. The process as claimed in claim 32, wherein the chlorinated hydrocarbon solvent is methylene dichloride.

40. The process as claimed in claim 32, wherein the hydrocarbon solvent is toluene.

41. The process as claimed in claim 32, wherein the rimonabant is dissolved at an elevated temperature.

42. The process as claimed in claim 32, wherein the rimonabant is dissolved at reflux temperature of the solvent used.

43. The process as claimed in claim 32, wherein the rimonabant is dissolved in ethyl acetate.

44. The process as claimed in claim 32, wherein the solvent is removed from the solution by vacuum drying or spray drying.

45. Crystalline rimonabant n-propanol solvate.

46. The compound as claimed in claim 45, wherein the crystalline rimonabant n-propanol solvate is characterized by an X-ray powder diffraction pattern having peaks expressed as 2θ at about 6.7, 8.3, 11.9, 13.4, 14.3, 15.9, 16.5, 17.9, 18.1, 19.2, 19.8, 20.5, 20.8, 21.4, 21.8, 22.2, 22.6, 24.1, 27.0 and 28.2±0.1 degrees.

47. A process for the preparation of crystalline rimonabant n-propanol solvate of claim 45, which comprises:
a) preparing a solution of rimonabant in n-propanol; and
b) isolating rimonabant n-propanol solvate from the solution obtained in step (a).

48. The process as claimed in claim 47, wherein the solution of rimonabant is prepared at an elevated temperature.

49. The process as claimed in claim 48, wherein the solution of rimonabant is prepared at reflux temperature.

50. The process as claimed in claim 47, wherein the isolation is initiated by cooling, seeding, partial removal of the solvent from the solution, by adding an anti-solvent to the solution or a combination thereof.

51. The process as claimed in claim 47, wherein the precipitated rimonabant n-propanol solvate crystals in step (b) are collected by filtration or centrifugation.

52. The process as claimed in claim 47, wherein the content of n-propanol in the crystalline rimonabant n-propanol solvate obtained is between 10% and 15% by weight.

53. Crystalline rimonabant n-butanol solvate.

54. The compound as claimed in claim 53, wherein the crystalline rimonabant n-butanol solvate is characterized by an X-ray powder diffraction pattern having peaks expressed as 2θ at about 7.5, 8.0, 9.1, 10.4, 16.1, 17.3, 22.4 and 23.8±0.1 degrees.

55. A process for the preparation of crystalline rimonabant n-butanol solvate of claim 53, which comprises:
a) preparing a solution of rimonabant in n-butanol; and
b) isolating rimonabant n-butanol solvate from the solution obtained in step (a).

56. The process as claimed in claim 55, wherein the solution of rimonabant is prepared at an elevated temperature.

57. The process as claimed in claim 56, wherein the solution of rimonabant is prepared at reflux temperature.

58. The process as claimed in claim 55, wherein the isolation is initiated by cooling, seeding, partial removal of the solvent from the solution, by adding an anti-solvent to the solution or a combination thereof.

59. The process as claimed in claim 55, wherein the precipitated rimonabant n-butanol solvate crystals in step (b) are collected by filtration or centrifugation.

60. The process as claimed in claim 56, wherein the content of n-butanol in the crystalline rimonabant n-butanol solvate obtained is between 10% and 15% by weight.

61. A process for the preparation of rimonabant crystalline Form II, which comprises:
a) preparing a solution of rimonabant in isopropyl alcohol; and
b) isolating rimonabant crystalline Form II from the solution obtained in step (a).

62. The process as claimed in claim 61, wherein the solution of rimonabant is prepared at an elevated temperature.

63. The process as claimed in claim 62, wherein the solution of rimonabant is prepared at reflux temperature.

64. The process as claimed in claim 61, wherein the isolation is initiated by cooling, seeding, partial removal of the solvent from the solution, by adding an anti-solvent to the solution or a combination thereof.

65. The process as claimed in claim 61, wherein the precipitated rimonabant form II crystals in step (b) are collected by filtration or centrifugation.

66. A pharmaceutical composition comprising crystalline rimonabant hydrate having a water content in the range of about 3-15% by weight of claim 1 and a pharmaceutically acceptable excipient.

67. The pharmaceutical composition as claimed in claim 66, wherein the pharmaceutical composition of crystalline rimonabant hydrate is a solid oral dosage form.

68. A pharmaceutical composition comprising crystalline rimonabant hydrate having a water content in the range of about 3-6% by weight of claim 1 and a pharmaceutically acceptable excipient.

69. The pharmaceutical composition as claimed in claim 68, wherein the pharmaceutical composition of crystalline rimonabant hydrate is a solid oral dosage form.

70. A pharmaceutical composition comprising amorphous rimonabant of claim 30 and a pharmaceutically acceptable excipient.

71. The pharmaceutical composition as claimed in claim 70, wherein the pharmaceutical composition of amorphous rimonabant is a solid oral dosage form.
